(19) United States
(12) Patent Application Publication
(10) Pub. No.: US 2024/0374377 A1
(43) Pub. Date: Nov. 14, 2024

(54) ANCHORS FOR IMPLANTABLE MEDICAL DEVICES

(71) Applicant: W. L. Gore & Associates, Inc., Newark, DE (US)

(72) Inventors: Kevin A. Gill, Flagstaff, AZ (US); Min-Hsuan Lin, Flagstaff, AZ (US); Rebecca L. Maryn, Flagstaff, AZ (US); David J. Minor, Phoenix, AZ (US); Edward E. Shaw, Flagstaff, AZ (US); Benjamin M. Trapp, Phoenix, AZ (US)

(21) Appl. No.: 18/686,791

(22) PCT Filed: Aug. 25, 2022
(86) PCT No.: PCT/US2022/041455

§ 371 Date: Feb. 26, 2024

Related U.S. Application Data
(60) Provisional application No. 63/237,902, filed on Aug. 27, 2021.

(51) Int. Cl. A61F 2/24; A61B 17/12; A61M 27/00

(52) U.S. Cl. CPC A61F 2/24; A61B 17/12122; A61B 17/12145; A61M 27/002; A61F 2220/0008; A61M 2210/125

(57) ABSTRACT

Various aspects of the present disclosure are directed toward apparatuses, systems, and methods that include an anchoring implantable medical device configured to anchor an implantable medical device.

### CROSS-REFERENCE TO RELATED APPLICATION

This application is a national phase application of PCT Application No. PCT/US2022/041455, internationally filed on Aug. 25, 2022, which claims the benefit of Provisional Application No. 63/237,902, filed Aug. 27, 2021, which are incorporated herein by reference in their entireties for all purposes.

### BACKGROUND

Implantable medical devices are often anchored in the body of a patient using an associated anchor. The anchor may be integral with other portions of the medical device, or may be a separate component that is attached to the medical device. Medical devices with which anchors are associated include grafts, stent grafts, pumps, or any of a variety of other medical devices that are to be held in place in the body. There are a variety of bodily structures to which medical devices may be anchored, such as the vasculature, organs, or other bodily structures. And, such medical devices may be configured to meet any of a variety of treatment objectives. For example, anchored medical devices may be utilized as occluders to treat atrial septal defects, patent forum ovale, or other conditions in intestines, fallopian tubes, or other bodily structures. Additional examples of anchored medical devices include those that help facilitate movement of fluids within a patient, such as shunts, prosthetic valves, and others. Still other types of implantable devices may include devices for monitoring and/or controlling physiologic conditions, such as smart medical devices.

### SUMMARY

According to one example, (“Example 1”) an anchor for an implantable medical device includes one or more wires arranged to form a first frame component and a second frame component. The first frame component includes a plurality of first contact features defining a plurality of first outer apices and at least one first inner apex arranged between each of the plurality of first contact features. The second frame component includes a plurality of second contact features defining a plurality of second outer apices and at least one second inner apex arranged between each of the plurality of second contact features. The one or more wires include a substantially linear section between each of the plurality of first outer apices and a substantially linear section between each of the plurality of second outer apices such that the plurality of first contact features each have approximately a diamond shape and the plurality of second contact features each have approximately a diamond shape.

According to another example, (“Example 2”), further to Example 1 the first plurality of contact features includes an odd number of contact features and the second plurality of contact features includes an odd number of contact features.

According to another example, (“Example 3”), further to Example 2, the first plurality of contact features includes five contact features and the second plurality of contact features includes five contact features.

According to another example, (“Example 4”), further to Examples 2 or 3, the first plurality of contact features and the second plurality of contact features are positioned opposite one another such that the first plurality of contact features and the plurality of second contact are angularly aligned with one another about a central longitudinal axis of the anchor.

According to another example, (“Example 5”), further to any one of Examples 1-4, the at least one first inner apex of each of the plurality of first contact features are adjacent to and spaced about a central longitudinal axis of the anchor, at least one second inner apex of each of the plurality of second contact features are adjacent to and spaced about the central longitudinal axis of the anchor.

According to another example, (“Example 6”), further to any one of Examples 1-5, the first and second frame components are connected by at least one turn of the one or more wires.

According to another example, (“Example 7”), further to any one of Examples 1-6, the anchor further comprises an intermediate portion extending between the first and second frame components.

According to another example, (“Example 8”), further to the Example of 7, the one or more wires includes at least one wire forming the first plurality of contact features, the intermediate portion, and the second plurality of contact features.

According to another example, (“Example 9”), further to Examples 7 or 8, the intermediate portion includes a membrane coupling the first frame component and the second frame component.

According to another example, (“Example 10”), further to any one of Examples 1 to 9, the first frame component and the second frame component are curved inwardly toward one another such that the plurality of first outer apices and the plurality of second outer apices are angled toward one another when the anchor is in an unconstrained state.

According to another example, (“Example 11”), further to any one of Examples 1 to 10, further comprising a membrane spanning areas of the first frame component and the second frame component defined by the one or more wires that form the first frame component and the second frame component.

According to another example, (“Example 12”), an implantable medical system includes an anchor that includes a wire. The wire forms an intermediate section, a first frame component and a second frame component. The first frame component is arranged on a first side of the intermediate portion and includes a plurality of first contact features each having a portion with an approximate diamond shape and the second frame component is arranged on a second side of the intermediate portion and includes a plurality of second contact features each having a portion with an approximate diamond shape.

According to another example, (“Example 13”), further to Example 12, the first frame component and the second frame component are curved inwardly toward one another such that the plurality of first outer apices and the plurality of second outer apices are angled toward one another when the anchor is in an unconstrained state.

According to another example, (“Example 14”), further to Example 13, the wire includes a continuous wind pattern forming the first frame component, the second frame component, and the intermediate portion.

According to another example, (“Example 15”), further to any one of Examples 12 to 14, the first frame component includes a plurality of first outer apices having curvatures and at least one first inner apex having a curvature arranged between each of the plurality of first contact features and the second frame component includes a plurality of second outer apices having curvatures and at least one first inner apex having a curvature arranged between each of the plurality of second contact features.

According to another example, (“Example 16”) a method of deploying an anchor for an implantable medical device includes arranging a first frame component of an anchor on a first side of a septum such that a plurality of first contact features of the first frame component contact the first side of the septum, the first frame component including one or more wound wires forming the plurality of first contact features, the plurality of first contact features including a substantially linear section between a plurality of first outer apices defined by the plurality of first contact features such that the plurality of first contact features each has a portion with approximately a diamond shape. A second frame component of the anchor is arranged on a second side of the septum such that a plurality of second contact features of the second frame component contact the second side of the septum.

According to another example, (“Example 17”), further to Example 16, the second frame component includes one or more wires and the plurality of second contact features include a substantially linear section between a plurality of second outer apices defined by the plurality of second contact features.

According to another example, (“Example 18”), further to Example 17, the plurality of second contact features each have a portion with approximately a diamond shape.

According to another example, (“Example 19”), further to Example 16, the first plurality of contact features includes an odd number of contact features and the second plurality of contact features includes an odd number of contact features.

According to another example, (“Example 20”), further to Example 19, the first frame component and the second frame component are curved inwardly toward one another such that the plurality of first outer apices and the plurality of second outer apices are angled toward one another when the anchor is in an unconstrained state.

According to another example, (“Example 21”), an anchor for anchoring an implantable medical device within a patient includes one or more wires forming a frame of the anchor. The anchor includes a first frame component including a plurality of first contact features including a plurality of first outer apices and a plurality of first inner apices arranged between each of the plurality of first contact features. The first frame component includes one or more wound wires forming the plurality of first contact features and the plurality of first contact features including a linear section between each of the plurality of first outer apices. The plurality of first contact features each has a portion approximating a diamond shape. And, a second frame component is arranged opposite the first frame component and includes a plurality of second contact features including a plurality of second outer apices and a plurality of second inner apices arranged between each of the plurality of second contact features.

According to another example, (“Example 22”), further to Example 21, the one or more wires are wound wires, where windings of the one or more wires form the first frame component and the second frame component.

According to another example, (“Example 23”), further to Examples 21 or 22, the anchor further comprises an intermediate portion between and connecting the first frame component and the second frame component, the intermediate portion including membrane material arranged between and connecting the first frame component and the second frame component.

According to another example, (“Example 24”), further to Example 23, one or more wires form the first frame component and one or more wires form the second frame component, the one or more wires that form the first frame component being separate from the one or more wires that form the second frame component.

According to another example, (“Example 25”), further to Examples 23 or 24, the intermediate portion is free of any wires.

According to another example, (“Example 26”), further to any one of Examples 21 to 25, the first frame component and the second frame component are curved inwardly toward one another such that the plurality of first outer apices and the plurality of second outer apices are angled toward one another when the anchor is in an unconstrained state.

According to another example, (“Example 26”), a system for anchoring an implantable medical device includes an anchor. The anchor includes a first frame component including a plurality of first contact features and a second frame component including a plurality of second contact features, the second frame component positioned opposite the first frame component. The anchor also includes an intermediate portion connecting the first frame component to the second frame component such that a longitudinal spacing between the first frame component and the second frame component is adjustable, the intermediate portion including a wound.

According to another example, (“Example 27”), an anchor for anchoring an implantable medical device within a patient includes one or more wires forming a frame of the anchor. The anchor includes a first frame component including a plurality of first contact features including a plurality of first outer apices and a plurality of first inner apices arranged between each of the plurality of first contact features, the first frame component including one or more wound wires forming the plurality of first contact features and the plurality of first contact features including a linear section between each of the plurality of first inner apices, the plurality of first contact features forming a plurality of first closed loops disposed about the central longitudinal axis of the anchor, each of the non-overlapping first closed loops being connected to two adjacent ones of the first closed loops by a plurality of first linear sections. The anchor also includes a second frame component arranged opposite the first frame component and including a plurality of second contact features including a plurality of second outer apices and a plurality of second inner apices arranged between each of the plurality of second contact features.

According to another example, (“Example 28”), further to Example 27, the one or more wound wires forming the plurality of second contact features and the plurality of second contact features include a linear section between each of the plurality of second inner apices, the plurality of second contact features forming a plurality of second closed loops disposed about the central longitudinal axis of the anchor, each of the non-overlapping second closed loops being connected to two adjacent ones of the second closed loops by a plurality of second linear sections.

According to another example, (“Example 29”), further to Examples 27 or 28, the plurality of first linear sections define an intermediate region positioned between the first frame component and the second frame component.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an example anchor that may be associated with an implantable medical device in accordance with an embodiment.

FIG. 2 is an example frame component that may form a part of an anchor that may be associated with an implantable medical device in accordance with an embodiment.

FIG. 3 is an example frame component that may form a part of an anchor that may be associated with an implantable medical device and a winding configuration in accordance with an embodiment.

FIG. 4 is an example anchor associated with an implantable medical device in accordance with an embodiment.

FIG. 5 is an example medical device that may be associated with an anchor in accordance with an embodiment.

FIG. 6 is another example anchor that may be associated with an implantable medical device in accordance with an embodiment.

FIG. 7 is another example anchor that may be associated with an implantable medical device in accordance with an embodiment.

FIG. 8 is another example anchor that may be associated with an implantable medical device in accordance with an embodiment.

FIG. 9 is another example anchor that may be associated with an implantable medical device in accordance with an embodiment.

FIG. 10 is an example anchor in accordance with an embodiment.

FIG. 11 is an example anchor and an implantable medical device in accordance with an embodiment.

FIG. 12 is an example anchor and an implantable medical device in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward anchoring an implantable medical device within a patient and medical devices for use within a patient. In certain instances, the embodiments of implantable medical device herein may be associated with (e.g., attached to and/or integrally formed with) an implantable medical device. The implantable medical device, which may be an occluder, may additionally be arranged with a shunt to treat heart failure when placed across an atrial septum, or any of a variety of other locations. These are examples only, and the anchors and various designs associated therewith may be used with such implantable medical devices implanted in a variety of locations in the body, such as shunts, prosthetic valves, and devices for monitoring and/or controlling physiologic conditions, such as smart medical devices, for example. The anchors, as discussed herein, may facilitate anchoring the medical device and minimize risk of embolization, minimize risk of creating loose thrombus, and/or minimize risk of enabling loose tissue growth—all of which can cause stroke. The apparatuses, systems, and methods are directed toward a catheter-delivered anchors for implantable medical devices or medical devices that anchor to structures in a patient.

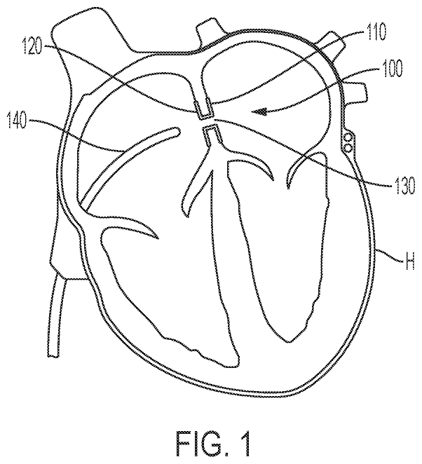

FIG. 1 is an anchoring implantable medical device in accordance with an embodiment. The anchor 100 is shown implanted within a heart H of a patient. The device 100 is shown arranged between the patient's left atrium and right atrium. In certain instances, the device 100 may be used to regulate blood pressure or pressures within the heart H, for example, between the left and right atria LA, RA. As shown, the device 100 generally includes a first frame component 110 arranged on a first side of a septum (e.g., within the right atrium RA), a second frame component 120 arranged on a second side of the septum (e.g., within the left atrium LA), and an intermediate section 130 extending through the septum. A valve may be placed within intermediate section 130 to assist in the regulation of blood pressures within the heart H. A needle may be used to create an opening in the septum. The designs may be used to anchor any of a variety of implantable medical devices at the location shown, including occluders, fenestrated occluders, and others. And, though the example of anchoring in a heart, and specifically a septal wall is depicted, as previously mentioned, any of a variety of locations within the body and associated treatments may benefit from the anchor designs disclosed herein.

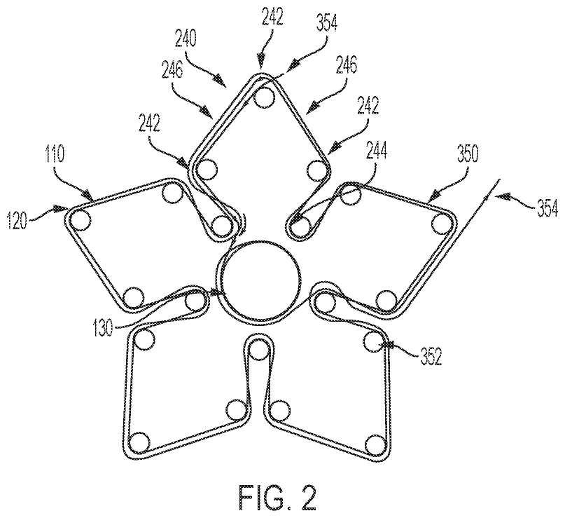

A sheath 140 and constraining and/or release lines (not shown) may be used to facilitate deployment of the device 100. For example, a first side of the device 100 that includes the first frame component 110 may be released after the sheath 140 is advanced through the septum and to the LA, and the second frame component 120 that includes the second frame component 120 may be released on the RA side of the septum. An intermediate section 130 (e.g., shown in FIG. 2) is arranged within the opening. The frame components 110, 120 and/or the intermediate section 130 may be compressed within the sheath 140 during delivery of the device 100 to the desired treatment area within the patient and subsequently expanded during deployment of the device 100. In other instances, the anchor 100 may be implanted at a different location in the heart or a different location within the patient (e.g., gastrointestinal system, vasculature, brain).

In certain instances, the intermediate section 130 is configured to be associated with at least a portion of an implantable medical device 500. The intermediate section 130 may be associated with (e.g., integrally formed with, attached, or otherwise associated with) the medical device 500 according to various examples. The intermediate section 130, in certain instances, may extend beyond each of the first frame component 110 and the second frame component 120 and between the first frame component 110 and the second first frame component 120 to facilitate anchoring of the implantable medical device 500 or to manage flow of blood, thrombus formation, or the like.

FIG. 2 shows an example winding pattern of first frame component 110 and second frame component 120. In particular, FIG. 2 shows two passes of a wire about a winding pattern to define the first frame component 110 and the second frame component 120, as well as engagement section 130 between the first and second frame components. The first frame component 110 is shown as the inner winding and the second frame component 120 is slightly offset and shown as an outer winding. This is done for illustrative purposes, as this is a two-dimensional representation of a three-dimensional object, and the windings may have essentially the same size according to various embodiments. And, although both the first and second frame components 110, 120 are shown, only one frame component may take the general shape shown in FIG. 2, and the other may be absent, or take a different shape according to various examples. As shown, a single wire 350 may be wound about a plurality of winding pegs 352 to form the first frame component 110 (and second frame component 120) as desired.

First frame component 110 (and second frame component 120) may form a part of an anchor for anchoring of an implantable medical device 500 in accordance with an embodiment. The shape shown for the first frame component 110 and/or the second frame component 120 may be the shape and configuration of the first frame component 110 and/or the second frame component 120 shown in FIGS. 1, 10, and 11. As shown, the first frame component 110 may include one or more wires, as can the second frame component 120. As described in further detail below, the one or more wires may form the first frame component 110, the second frame component 120, and an intermediate section 130 between the first and second frame components (e.g., as shown in FIGS. 1, 10, and 11) of an anchor. The first frame component 110 may be arranged on a first side of the intermediate section 130 and the second frame component 120 may be arranged on a second side of the intermediate section 130 so that the intermediate section 130 is between or substantially between the frame components.

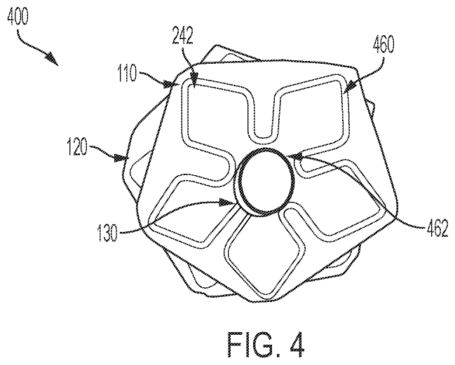

Each of the first frame component 110 and the second frame component 120 may include a plurality of contact features 240, one location for which is highlighted in FIG. 2 for ease of illustration. Contact features are suitable for contact with structures within the patient. Embodiments with contact features may be configured to allow tissue ingrowth to enhance long term anchoring and to be atraumatic to the tissue they contact to reduce incidences of tissue erosion, which in some embodiments may be accomplished by covering contact features with films. For example, a membrane 460, also described as a covering, may be arranged across the contact features 242 of the first frame component 110 and the contact features 242 of the second frame component 120 as shown and subsequently described in association with FIG. 4.

In instances where an anchor includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and at least one inner apex 244 arranged between each of the plurality of contact features 240. In instances where an anchoring device includes the first frame component 110 and the second frame component 120, the plurality of outer apices 242 and the at least one inner apex 244 of the first frame component 110 may be a first plurality of outer apices 242 and at least one first inner apex 244 and the plurality of outer apices 242 at least one inner apex 244 of the second frame component 120 may be a plurality of second outer apices 242 and at least one second inner apex 244.

In certain instances, the one or more wires that form the frame component 100 include a substantially linear section 246 between each of the plurality of outer apices 242. The substantially linear section 246 between each of the plurality of outer apices 242 may form approximately a diamond shape of the contact features 242 as is shown in FIG. 2. In addition, the plurality of contact features 242 may be an odd number of contact features 242 (e.g., three, five, seven) in certain instances or an even number of contact features 242 (e.g., two, four, six, eight) in other instances. In certain instances, the at least one inner apex 244 of each of the plurality of contact features 242 is adjacent to and spaced about a circumference of the intermediate section 130. In certain instances, the outer apices 242 and at least one inner apex 244 have curvatures.

In certain instances, the plurality of outer apices 242 may be used to engage deployment tethers or portions of a deployment system. The plurality of outer apices 242 may also engage retrieval loops to allow for recapture or retrieval of the device after it is deployed. The deployment system may use one or more constraining lines and/or release lines as discussed in further detail in WO2020018697A1 (Cole et al.) which is incorporated herein.

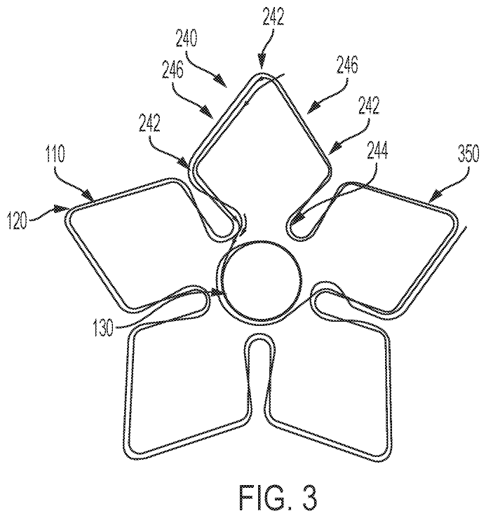

FIG. 3 shows the first frame component 110 and the second frame component 120 that may form a part of an anchor configured to anchor an implantable medical device and a winding configuration in accordance with an embodiment. In FIG. 3, the winding pegs 352 are not shown, and the ends of the wire 350 have been trimmed to the final frame shape. Though one wire is shown in FIGS. 2 and 3, the first frame component 110 may be formed of one or more wires 350.

As shown in FIG. 2, in certain instances, the wire(s) 350 may including a starting point 354 and an ending point 354 in forming a first frame component 110. In certain instances, the starting point 354 of the wire(s) 350 may be within the first frame component 110 and the ending point 354 may be within a second frame component 120. In addition, the wire(s) 350 may form the intermediate section 130. In certain instances, the intermediate section 130 includes a circumference that includes at least one turn of the wire(s) 350. The wire(s) 350 may include at least one wire 350 forming the first plurality of contact features 242, the section 130, and the second plurality of contact features 242. The wire(s) 350 includes a continuous wind pattern in forming the first frame component 110, the second frame component 120, and the intermediate section 130.

As shown, the first frame component 110 includes five contact features 242 each of which include a diamond shape. In certain instances, the five contact features 242 (or an odd number of contact features) may resist prolapse when implanted in the atrial septum (or target area of the body/patient). The shape and arrangement of the contact features 242 may resist prolapse and stabilize an implantable medical device 500 arranged within the intermediate section 130. The odd number of contact features 242 and shape of the contact features may facilitate crush or constraining of the contact features 242 while also minimizing stress, minimizing prolapse, and allowing for retrievability. In certain instances, the frame components 110, 120 may be substantially planar or may be concave or convex.

FIG. 4 is an example anchor 400 for an implantable medical device in accordance with an embodiment. The anchor 400 may be part of a system that includes an implantable medical device 500 (sensor, pump, lead, or other medical device). As discussed in detail above, the anchor 400 includes an intermediate section 130 configured to allow placement of an implantable medical device 500 within the section 130, a first frame component 110 arranged on a first side of the intermediate section 130, which may include an odd number of contact features 242 each having a portion with an approximate diamond shape, and a second frame component 120 arranged on a second side of the intermediate section 130, which may include an odd number of contact features 242 each having a portion with an approximate diamond shape.

In certain instances, the first frame component 110, the second frame component 120, and the intermediate section 130 are formed by at least one wire. As shown in FIGS. 2 and 3, the wire(s) 350 includes a continuous wind pattern in forming the first frame component 110, the second frame component 120, and the intermediate section 130. In certain instances, the first frame component 110 and the second frame component 120 are formed from separate wires with separate membranes. The first frame component 110 and the second frame component 120 may be separate from one another. In other instances, the intermediate section 130 may be formed of a membrane that separates the first frame component 110 and the second frame component 120. In addition, one or both of the first frame component 110 and the second frame component 120 may be concave.

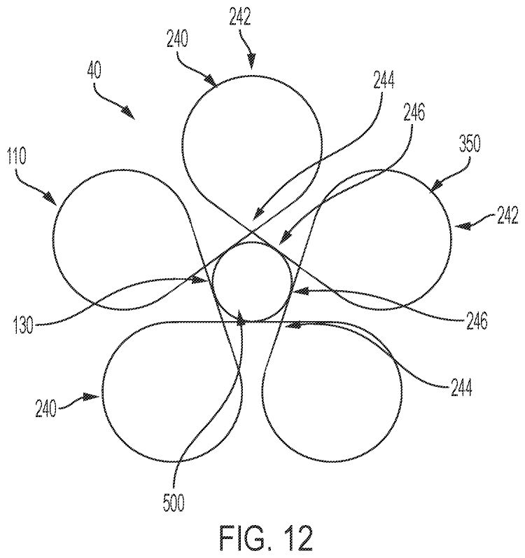

In certain instances, and as is shown in FIG. 4, the contact features 242 of the first frame component 110 and the contact features 242 of the second frame component 120 are aligned (e.g., relative to a longitudinal axis of the intermediate section 130). The contact features 242 of each of the frame components 110, 120 being allowed may allow for crossing or re-crossing of the anchor 400 after implantation. In certain instances, the anchor 400 includes a membrane 460 coupled to the first frame component 110 and the second frame component 120. The membrane 460 may be arranged across the contact features 242 of the first frame component 110 and the contact features 242 of the second frame component 120. As shown in FIG. 4, the membrane 460 spans areas 462 of the first frame component 110 and the second frame component 120 that do not include the at least one wire that forms the first frame component 110 and the second frame component 120. As noted above, the contact features 242 of the first frame component 110 and the contact features 242 of the second frame component 120 are aligned. The areas 462 of the membrane where there are no wire(s) may be punctured for re-crossing (e.g., for a future procedure such as ablation, left atrial appendage device implantation). Similar concepts apply to the designs shown in FIGS. 2 and 12, where the membrane 460 may be included and cover the contact features 242 of those designs. And, similarly there, the membrane where there are no wire(s) may be punctured for re-crossing (e.g., for a future procedure such as ablation, left atrial appendage device implantation)

In implanting the anchor 400, which may be used in a method of managing treatment of a patient with congestive heart failure, occluding a defect in the wall of a body structure, or for other treatment as desired. As shown, the anchor 400 may be placed on a tissue (e.g., atrial septum) of the patient. The first frame component 110 may be arranged on a first side of the septum and the second frame component 120 may be arranged on a second side of the septum. After deployment of the anchor 400, an implantable medical device 500 may be arranged within intermediate section 130 or the opening defined by intermediate section 130 in the embodiments may be increased to allow blood flow to treat heart failure or the opening defined by intermediate section 130 may be decreased such that blood flow would not occur and a defect is occluded. In other instances, the implantable medical device 500 may be built with the anchor 400 and delivered in place.

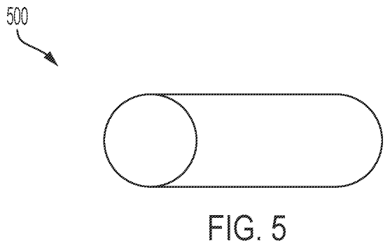

FIG. 5 includes a generalized view of an implantable medical device 500 that may be coupled to the anchor 400 and anchored within the body of a patient. The implantable medical device 500 may be any of a variety of devices, such as a stent graft, stent, valve, pump, lead, or other type of medical device including smart medical devices with health monitoring capabilities or the like.

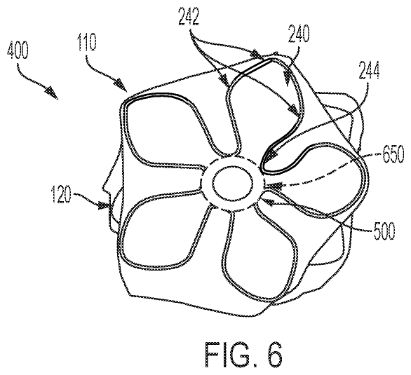

FIG. 6 is another example anchor 400 for anchoring an implantable medical device in accordance with an embodiment or for treatment of a condition in a patient such as occluding a defect. The anchor 400 may anchor a pump, lead, or other type of medical device that can be anchored within a patient. The first frame component 110 and second frame component 120 are shown in FIG. 6, where the features of the second frame component 120 are partially hidden by the first frame component 110. It should also be clear that the second frame component 120 may have similar features to those shown for the first frame component 110. The first frame component 110 may include one or more wires. The one or more wires may form the first frame component 110, the second frame component 120, and an intermediate portion (e.g., intermediate section 130 shown in FIG. 1). In other instances, the intermediate section 130 may include a graft material arranged between and connecting the first frame component 110 and the second frame component 120. The first frame component 110 may be arranged on a first side of the intermediate portion and the second frame component 120 may be arranged on a second side of the intermediate portion.

The first frame component 110 may include a plurality of contact features 240, one of which is highlighted in FIG. 6 for ease of illustration. In instances where an implantable device includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and a plurality of inner apices 244 arranged between each of the plurality of contact features 240. In instances where an implantable device includes the first frame component 110 and the second frame component 120, the plurality of outer apices 242 and the plurality of inner apices 244 of the first frame component 110 may be a first plurality of outer apices 242 and the plurality of inner apices 244 and the plurality of outer apices 242 and the plurality of inner apices 244 of the second frame component 120 may be a plurality of second outer apices 242 and a plurality of second inner apices 244.

The inner apices 244 may define inner engagement features for engagement with one or more features of a medical device. In certain instances, and as is shown, the plurality of first inner apices 244 and/or the plurality of second inner apices 244 may be configured to anchor an implantable medical device 500. The anchor 400 may be associated with the implantable device in a variety of ways, including via the intermediate section 130, also described as an intermediate portion or central portion of the anchor. The shape of the intermediate section 130 may take on a variety of shapes for a variety of purposes, including anchor performance, ease of manufacture, and other functions. For example, the shape of the intermediate section 130 may promote better flexing and/or fatigue reliability, may facilitate association with medical devices of differing configurations, or others.

In certain instances, the portions of the one or more wires that form the first frame component 110 are uncoupled from portions of the one or more wires that form the second frame component 120 are unconnected.

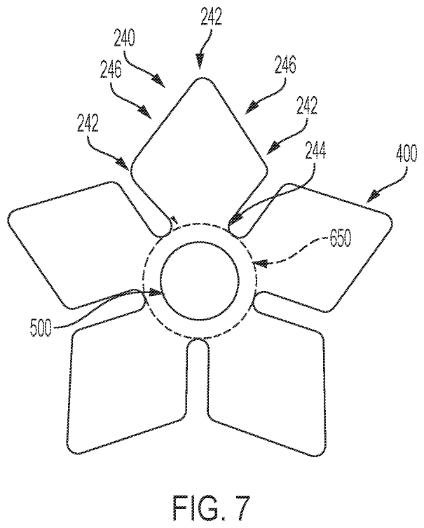

FIG. 7 is another example anchor 400. The anchor 400 may anchor a pump, lead, or other type of medical device that can be anchored within a patient. The first frame component 110 shown in FIG. 6 may be the first frame component 110 and/or the second frame component 120 (e.g., as shown in FIGS. 1, 10, and 11). The first frame component 110 may include one or more wires. The one or more wires may form the first frame component 110, the second frame component 120, and an intermediate portion (e.g., engagement section shown in FIG. 1). A membrane may cover the first frame and second frame components. In other instances, the intermediate portion may include a graft material arranged between and connecting the first frame component 110 and the second frame component 120. The first frame component 110 may be arranged on a first side of the intermediate portion and the second frame component 120 may be arranged on a second side of the intermediate portion.

The first frame component 110 may include a plurality of contact features 240, one of which is highlighted in FIG. 7 for ease of illustration. In instances where an implantable device includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and a plurality of inner apices 244 arranged between each of the plurality of contact features 240. In instances where an implantable device includes the first frame component 110 and the second frame component 120, the plurality of outer apices 242 and the plurality of inner apices 244 of the first frame component 110 may be a first plurality of outer apices 242 and the plurality of inner apices 244 and the plurality of outer apices 242 and the plurality of inner apices 244 of the second frame component 120 may be a plurality of second outer apices 242 and a plurality of second inner apices 244.

In certain instances, the portions of the one or more wires that form the first frame component 110 are uncoupled from portions of the one or more wires that form the second frame component 120 and are unconnected. In addition, the first frame component 110 and the second frame component 120 may be separated by an intermediate portion (e.g., as shown in FIGS. 1, 10, and 11). The intermediate portion may be formed by portions of the wires and/or by a graft material arranged between and connecting the first frame component 110 and the second frame component 120.

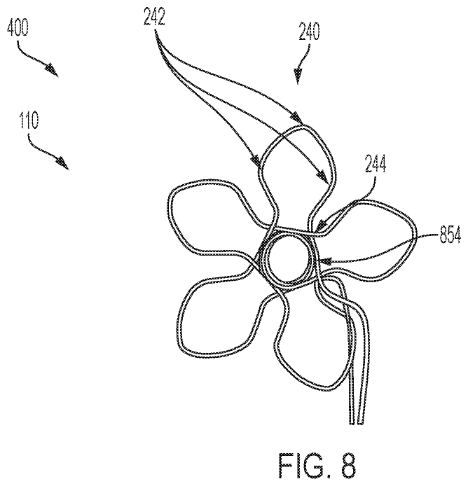

FIG. 8 is another example implantable medical device including an anchor 400 in accordance with an embodiment. The anchor 400 may anchor a pump, lead, or other type of medical device that can be anchored within a patient. The first frame component 110 shown in FIG. 8 may be the first frame component 110 and/or the second frame component 120 (e.g., as shown in FIGS. 1, 10, and 11). The first frame component 110 may include one or more wires. The one or more wires may form the first frame component 110, the second frame component 120, and an intermediate portion (e.g., engagement section shown in FIG. 1). In other instances, the intermediate portion may include a graft material arranged between and connecting the first frame component 110 and the second frame component 120. The first frame component 110 may be arranged on a first side of the intermediate portion and the second frame component 120 may be arranged on a second side of the intermediate portion.

The first frame component 110 may include a plurality of contact features 240, one of which is highlighted in FIG. 8 for ease of illustration. In instances where an implantable device includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and a plurality of inner apices 244 arranged between each of the plurality of contact features 240. In instances where an implantable device includes the first frame component 110 and the second frame component 120, the plurality of outer apices 242 and the plurality of inner apices 244 of the first frame component 110 may be a first plurality of outer apices 242 and the plurality of inner apices 244 and the plurality of outer apices 242 and the plurality of inner apices 244 of the second frame component 120 may be a plurality of second outer apices 242 and a plurality of second inner apices 244.

In certain instances, the portions of the one or more wires that form the first frame component 110 are uncoupled from portions of the one or more wires that form the second frame component 120 and are unconnected. For example, the one or more wires may form a first section 854 forming a boundary extending between the plurality of first inner apices 244 configured to connect to the implantable medical device 500 within the first frame component 110. The second frame component 120 may also include a second section 854 formed by portions of the one or more wire that may also be configured to connect to the implantable medical device 500 within the second frame component 120. In certain instances, the first inner engagement section 854 and/or the second inner engagement section 854 may have approximately a circular shape or other shape to conform to the outer shape of the medical device to be anchored. The first section 854 and/or the second section 854 may be configured to limit rotational freedom of the implantable medical device 500 when anchored.

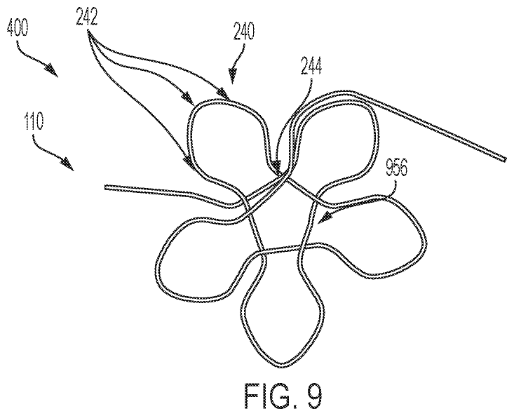

FIG. 9 is another example implantable anchor for a medical device in accordance with an embodiment. The anchor 400 may anchor a pump, lead, or other type of medical device that can be anchored within a patient. The first frame component 110 shown in FIG. 9 may be the first frame component 110 and/or the second frame component 120 (e.g., as shown in FIGS. 1, 10, and 11). The first frame component 110 may include one or more wires. The one or more wires may form the first frame component 110, the second frame component 120, and an intermediate portion (e.g., intermediate section 130 shown in FIG. 1). In other instances, the intermediate portion may include a graft material arranged between and connecting the first frame component 110 and the second frame component 120. The first frame component 110 may be arranged on a first side of the intermediate portion and the second frame component 120 may be arranged on a second side of the intermediate portion.

The first frame component 110 may include a plurality of contact features 240, one of which is highlighted in FIG. 9 for ease of illustration. In instances where an implantable device includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and a plurality of inner apices 244 arranged between each of the plurality of contact features 240. In instances where an implantable device includes the first frame component 110 and the second frame component 120, the plurality of outer apices 242 and the plurality of inner apices 244 of the first frame component 110 may be a first plurality of outer apices 242 and the plurality of inner apices 244 and the plurality of outer apices 242 and the plurality of inner apices 244 of the second frame component 120 may be a plurality of second outer apices 242 and a plurality of second inner apices 244.

In certain instances, the portions of the one or more wires that form the first frame component 110 are uncoupled from portions of the one or more wires that form the second frame component 120 are unconnected. In addition, the one or more wires may also form a first section 854 forming a boundary extending between the plurality of first inner apices 244. The second frame component 120 may also include a second section 854 formed by portions of the one or more wires. The first section 854 and/or the second section 854 may be configured to be associated with an implantable medical device. In certain instances, the first section 854 and/or the second section 854 may have approximately a polygonal shape.

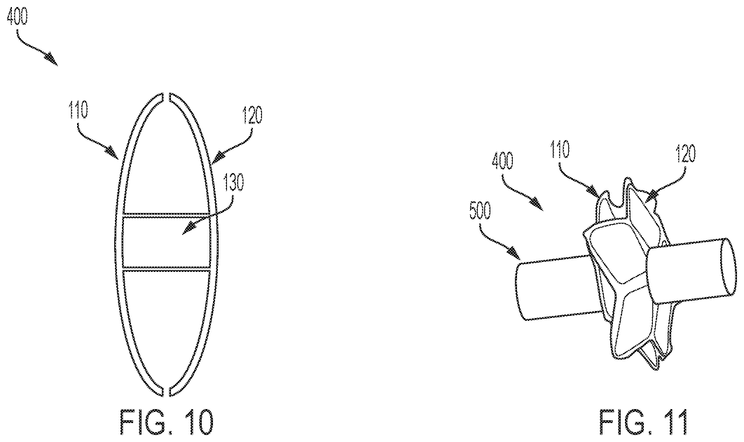

FIGS. 10 and 11 show a configuration in which the first frame component 110 and the second frame component 120 are curved (e.g., convexly curved), or are otherwise curved toward one another when the anchor is in an unbiased, or unconstrained state. FIG. 10 is a schematic representation of this feature, and FIG. 11 shows one example of an anchor 400 so configured. In certain instances, the first frame component 110 and the second frame component 120 are curved inwardly relative to the intermediate section 130. The curved frame components 110, 120 may facilitate enhanced contact with a target tissue location, lessening of the opportunity for obstructive tissue growth along the curved frame components 110, 120 that could interfere with implantable medical device 500 performance, and/or reduce or avoid other detrimental effects, such as thrombus formations, and others. In some examples, the curvature of the anchor frame components 110, 120 enhances apposition against an interposed bodily structure (e.g., tissue), providing better potential contact of outer apices 242 without unwanted spacing between the contact features 240 and the interposed structure (e.g., without “coning” or “tenting” of the anchor 400. In some examples, the curved frame components 110, 120 are configured such that the plurality of first outer apices 242 and the plurality of second outer apices 242 are angled toward one another when the anchor 400 is in an unconstrained state.

FIG. 12 shows still another configuration of anchor 400 according to various embodiments. As shown, the anchor 400 includes at least one wire 350 defining a plurality of contact features 240 in the form closed wire loops, where the wire loops close by overlapping at the inner apices 244. Only the first frame component 110 is visible in FIG. 12, but the second frame component 120 may have substantially the same configuration, or may differ either in size or shape, or in orientation (e.g., by being rotationally offset from the first frame component 110). The contact features 240 define a plurality of outer apices 242. Each of the inner apices 244 of the contact features 240 are connected to two adjacent ones of the inner apices 244. As shown, they are connected by a linear section 246 extending between each of the plurality of outer apices 242. This plurality of linear sections 246 may define the intermediate section 130, as shown, which is associated with implantable medical device 500. Also, as shown, the closed wire loops defined by the contact features 240 are positioned outside, and do not overlap with one another outside of the inner apices 244, nor do they overlap inside of the intermediate section 130. In other words, the plurality of contact features 240 define a plurality of non-overlapping closed loops disposed about the central longitudinal axis of the anchor 400, each of the non-overlapping closed loops being connected to two adjacent ones of the closed loops by a plurality of linear sections 246.

With the foregoing in mind, an anchor 400 according to the design of FIG. 12 may include first frame component 110 including plurality of first contact features 240 including plurality of first outer apices 242 and plurality of first inner apices 244 arranged between each of the plurality of first contact features 240. The first frame component 110 may include one or more wound wires 350 forming the plurality of first contact features 240 and the plurality of first contact features may include a plurality of linear sections 246 between each of the plurality of first inner apices 244. The plurality of first contact features 240 may forming a plurality of first closed loops disposed about the central longitudinal axis of the anchor, each of the non-overlapping first closed loops being connected to two adjacent ones of the first closed loops by a plurality of first linear sections 246.

Second frame component 120 may be similarly configured, with the one or more wound wires 350 forming plurality of second contact features 240 including second linear sections 356 between each of the second inner apices 244, the plurality of second contact features 240 forming a plurality of second closed loops disposed about the central longitudinal axis of the anchor, each of the non-overlapping second closed loops being connected to two adjacent ones of the second closed loops by a plurality of second linear sections 246.

And, as previously referenced, the plurality of first linear sections 246 (and/or the plurality of second linear sections 246) may define intermediate region 130 between the first frame component 110 and the second frame component 120.

A membrane material, discussed herein, may include a fluoropolymer, such as a polytetrafluoroethylene (PTFE) polymer or an expanded polytetrafluoroethylene (ePTFE) polymer. In some instances, the membrane material may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the membrane material can include Dacron, polyolefins, carboxy methylcellulose fabrics, polyurethanes, or other woven, non-woven, or film elastomers.

In addition, nitinol (NiTi) may be used as the material of the frame (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the frame elements. In addition, NiTi can be shape-set into a desired shape. That is, NiTi can be shape-set so that the frame tends to self-expand into a desired shape when the frame is unconstrained, such as when the frame is deployed out from a delivery system.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An anchor for an implantable medical device, the anchor comprising:
one or more wires arranged to form:
a first frame component including a plurality of first contact features defining a plurality of first outer apices and at least one first inner apex arranged between each of the plurality of first contact features; and
a second frame component including a plurality of second contact features defining a plurality of second outer apices and at least one second inner apex arranged between each of the plurality of second contact features,
the one or more wires including a substantially linear section between each of the plurality of first outer apices and a substantially linear section between each of the plurality of second outer apices such that the plurality of first contact features each have approximately a diamond shape and the plurality of second contact features each have approximately a diamond shape.

2. The anchor of claim 1, wherein the first plurality of contact features includes an odd number of contact features and the second plurality of contact features includes an odd number of contact features.

3. The anchor of claim 2, wherein the first plurality of contact features includes five contact features and the second plurality of contact features includes five contact features.

4. The anchor of claim 2, wherein the first plurality of contact features and the second plurality of contact features are positioned opposite one another such that the first plurality of contact features and the plurality of second contact are angularly aligned with one another about a central longitudinal axis of the anchor.

5. The anchor of claim 1, wherein the at least one first inner apex of each of the plurality of first contact features are adjacent to and spaced about a central longitudinal axis of the anchor, at least one second inner apex of each of the plurality of second contact features are adjacent to and spaced about the central longitudinal axis of the anchor.

6. The anchor of claim 1, wherein the first and second frame components are connected by at least one turn of the one or more wires.

7. The anchor of claim 1, wherein the anchor further comprises an intermediate portion extending between the first and second frame components.

8. The anchor of claim 7, wherein the one or more wires includes at least one wire forming the first plurality of contact features, the intermediate portion, and the second plurality of contact features.

9. The anchor of claim 7, wherein the intermediate portion includes a membrane coupling the first frame component and the second frame component.

10. The anchor of claim 1, wherein the first frame component and the second frame component are curved inwardly toward one another such that the plurality of first outer apices and the plurality of second outer apices are angled toward one another when the anchor is in an unconstrained state.

11. The anchor of claim 1, further comprising a membrane spanning areas of the first frame component and the second frame component defined by the one or more wires that form the first frame component and the second frame component.

12. An implantable medical system comprising:
an anchor that includes a wire that forms:
an intermediate section;
a first frame component arranged on a first side of the intermediate portion and including a plurality of first contact features each having a portion with an approximate diamond shape; and
a second frame component arranged on a second side of the intermediate portion and including a plurality of second contact features each having a portion with an approximate diamond shape.

13. The implantable medical system of claim 12, wherein the first frame component and the second frame component are curved inwardly toward one another such that the plurality of first outer apices and the plurality of second outer apices are angled toward one another when the anchor is in an unconstrained state.

14. The implantable medical system of claim 13, wherein the wire includes a continuous wind pattern forming the first frame component, the second frame component, and the intermediate portion.

15. The implantable medical system of claim 12, wherein the first frame component includes a plurality of first outer apices having curvatures and at least one first inner apex having a curvature arranged between each of the plurality of first contact features and the second frame component includes a plurality of second outer apices having curvatures and at least one first inner apex having a curvature arranged between each of the plurality of second contact features.

16. A method of deploying an anchor for an implantable medical device, the method comprising:
arranging a first frame component of an anchor on a first side of a septum such that a plurality of first contact features of the first frame component contact the first side of the septum, the first frame component including one or more wound wires forming the plurality of first contact features, the plurality of first contact features including a substantially linear section between a plurality of first outer apices defined by the plurality of first contact features such that the plurality of first contact features each has a portion with approximately a diamond shape;
arranging a second frame component of the anchor on a second side of the septum such that a plurality of second contact features of the second frame component contact the second side of the septum.

17. The method of claim 16, wherein the second frame component includes one or more wires and the plurality of second contact features include a substantially linear section between a plurality of second outer apices defined by the plurality of second contact features.

18. The method of claim 17, wherein the plurality of second contact features each have a portion with approximately a diamond shape.

19. The method of claim 16, wherein the first plurality of contact features includes an odd number of contact features and the second plurality of contact features includes an odd number of contact features.

20. The method of claim 19, wherein the first frame component and the second frame component are curved inwardly toward one another such that the plurality of first outer apices and the plurality of second outer apices are angled toward one another when the anchor is in an unconstrained state.

21. An anchor for anchoring an implantable medical device within a patient, the anchor including one or more wires forming a frame of the anchor, the anchor comprising:
a first frame component including a plurality of first contact features including a plurality of first outer apices and a plurality of first inner apices arranged between each of the plurality of first contact features, the first frame component including one or more wound wires forming the plurality of first contact features and the plurality of first contact features including a linear section between each of the plurality of first outer apices, the plurality of first contact features each having a portion approximating a diamond shape; and
a second frame component arranged opposite the first frame component and including a plurality of second contact features including a plurality of second outer apices and a plurality of second inner apices arranged between each of the plurality of second contact features.

22. The anchor of claim 21, wherein the one or more wires are wound wires, where windings of the one or more wires form the first frame component and the second frame component.

23. The anchor of claim 21, further comprising an intermediate portion between and connecting the first frame component and the second frame component, the intermediate portion including membrane material arranged between and connecting the first frame component and the second frame component.

24. The anchor of claim 23, wherein one or more wires form the first frame component and one or more wires form the second frame component, the one or more wires that form the first frame component being separate from the one or more wires that form the second frame component.

25. The anchor of claim 23, wherein the intermediate portion is free of any wires.

26. The anchor of claim 21, wherein the first frame component and the second frame component are curved inwardly toward one another such that the plurality of first outer apices and the plurality of second outer apices are angled toward one another when the anchor is in an unconstrained state.

26. A system for anchoring an implantable medical device, the system comprising:
an anchor that includes:
a first frame component including a plurality of first contact features; and
a second frame component including a plurality of second contact features, the second frame component positioned opposite the first frame component; and
an intermediate portion connecting the first frame component to the second frame component such that a longitudinal spacing between the first frame component and the second frame component is adjustable, the intermediate portion including a wound.

27. An anchor for anchoring an implantable medical device within a patient, the anchor including one or more wires forming a frame of the anchor, the anchor comprising:
a first frame component including a plurality of first contact features including a plurality of first outer apices and a plurality of first inner apices arranged between each of the plurality of first contact features, the first frame component including one or more wound wires forming the plurality of first contact features and the plurality of first contact features including a linear section between each of the plurality of first inner apices, the plurality of first contact features forming a plurality of first closed loops disposed about the central longitudinal axis of the anchor, each of the non-overlapping first closed loops being connected to two adjacent ones of the first closed loops by a plurality of first linear sections; and
a second frame component arranged opposite the first frame component and including a plurality of second contact features including a plurality of second outer apices and a plurality of second inner apices arranged between each of the plurality of second contact features.

28. The anchor of claim 27, wherein the one or more wound wires forming the plurality of second contact features and the plurality of second contact features include a linear section between each of the plurality of second inner apices, the plurality of second contact features forming a plurality of second closed loops disposed about the central longitudinal axis of the anchor, each of the non-overlapping second closed loops being connected to two adjacent ones of the second closed loops by a plurality of second linear sections.

29. The anchor of claim 27, where the plurality of first linear sections define an intermediate region positioned between the first frame component and the second frame component.
